(19) United States
(12) Patent Application Publication
(10) Pub. No.: US 2015/0050707 A1
(43) Pub. Date: Feb. 19, 2015

(54) TREATMENT OF BIOMASS

(71) Applicant: TERAX Limited Partnership, Rotorua (NZ)

(72) Inventors: Daniel James Gapes, Rotorua (NZ); Trevor Raymond Stuthridge, Rotorua (NZ); Peter James Strong, Rotorua (NZ); Robert Jason Lei, Rotorua (NZ); Anderson Aggrey, Rotorua (NZ)

(21) Appl. No.: 14/381,503

(22) PCT Filed: Feb. 27, 2014
(86) PCT No.: PCT/IB2013/051579

§ 371 Date: Aug. 27, 2014

(30) Foreign Application Priority Data
Feb. 28, 2012 (NZ) 598479

(51) Int. Cl. C12P 7/64; C07C 29/50; C12P 7/52; C07C 51/21; C12P 7/54; C12P 7/04

(52) U.S. Cl. CPC C12P 7/6409; C12P 7/54; C12P 7/04; C12P 7/52; C07C 51/21; C07C 29/50
USPC 435/134; 435/150; 435/157; 435/140; 435/161; 435/160; 435/163; 435/165; 435/141

(57) ABSTRACT

A process for the treatment of biomass comprising subjecting biomass to microbial digestion to produce volatile fatty acids and/or solvents followed by wet oxidation to reduce biosolid volume while retaining or increasing the concentration of the volatile fatty acids and/or solvents.

### FIELD OF INVENTION

The present invention relates to a process for the treatment of biomass, particularly biological waste material such as municipal waste water, and transforming at least part of the biomass into separable output streams. In particular, the present invention relates to a process for the treatment of biomass comprising subjecting biomass to microbial digestion to produce volatile fatty acids and/or solvents followed by wet oxidation to reduce biosolid volume while retaining or increasing the concentration of the volatile fatty acids and/or solvents.

### BACKGROUND

Treatment of biological wastes such as municipal biosolids is necessary to achieve a number of desirable endpoints, including remediation of the water content, reduction of the volume of solids (sludge) that must be disposed of, increasing the biodegradability of any solids, and reducing the toxicity of any residue. Ideally the treatment process will generate one or more desirable by-products, including clean water, energy, fertiliser, fuel or fuel components and useful chemicals.

A common treatment process involves a sedimentation step followed by treatment with aerobic microorganisms combined with numerous other treatment and separation methods such as filtration, nutrient removal and others. Such processes generally produce large quantities of sludge that often require further treatment and disposal in landfills or at sea, or incineration.

A number of methods have been reported to reduce sludge volume including addition of oxygen gas, autothermal aerobic digestion, anaerobic digestion and the addition of oxidising agents. Wet oxidation has been demonstrated as an effective but expensive method of reducing the volume of sludge output from municipal waste water treatment plants with the destruction of almost all of the organic material by oxidation to CO₂ leaving a relatively small volume of recalcitrant (mostly mineral) material behind.

Known waste treatment plants using microbial digestion or wet oxidation or combinations of the two are almost solely directed to the destruction of the biomass to reduce the need for land-filling or incineration.

Microbial digestion processes for municipal waste biosolids generally use acidogenesis and acetogenesis (producing volatile fatty acids) followed by methanogenesis to break down the waste to methane. Such processes leave a large proportion of the incoming waste as sludge and volatile fatty acids are lost through conversion into methane by methanogenic microorganisms.

Anaerobic digestion is used to produce acetate/acetic acid for uses including as a feedstock in the production of hydrogen gas via gasification and alcohols although often this is generated from cleaner feedstocks such as lignocellulosic biomass where biomass destruction is not the primary objective.

Microbial digestion has advantages over wet oxidation for the production of useful carbon by-products insofar as it can be adapted to produce a wider range of carbon based molecules including volatile fatty acids and alcohols and acetone.

By contrast, wet oxidation processes can achieve a much greater reduction in biosolids leaving only a small recalcitrant fraction of mostly mineral composition but yields of useful carbon by-products are small. Known wet oxidation treatment processes for waste water are primarily directed to destruction of the waste and oxidise the vast majority of the biomass to CO₂ which is discharged to the atmosphere.

When wet oxidation is directed to production of volatile fatty acids and applied to waste water the yields of volatile fatty acids and the like are typically only in the 10-15% range. Achieving yields above this is extremely difficult.

For operative and capital cost reasons some waste treatment plants combine lower cost microbial digestion followed by wet oxidation to treat the waste components that are largely resistant to microbial digestion. However, as stated above the known processes are directed primarily to destruction to methane (in the microbial stage) and CO₂ (in the wet air oxidation stage).

It is an object of the invention to provide an improved or alternative process for treatment of waste effluents containing organic material to result in a reduction of sludge volume and the production of useful chemical by-products.

### SUMMARY OF THE INVENTION

In broad terms the present invention generally relates to a process for the treatment of biomass comprising subjecting biomass to microbial digestion, preferably anaerobic microbial digestion to produce volatile fatty acids and/or solvents followed by wet oxidation to reduce biosolid volume while retaining or increasing the concentration of the volatile fatty acids and/or solvents. For example, the process may comprise

(1) subjecting biomass to microbial digestion, preferably anaerobic microbial digestion under conditions so as to convert at least a portion of the organic biomass to volatile fatty acids and/or solvents while leaving at least some of the organic biomass in the form of biosolids or unconverted organic material to create a mixture of biosolids, unconverted organic biomass and volatile fatty acids and/or solvents, and

(2) subjecting the mixture to wet oxidation thereby reducing biosolid volume and producing a resulting mixture under conditions that do not result in the mass destruction of the volatile fatty acids and/or solvents.

In one aspect the present invention relates to a process for the treatment of biomass comprising

(1) subjecting biomass to microbial digestion, preferably anaerobic microbial digestion by contacting biomass with one or more microorganisms under conditions that promote acidogenesis while retarding methanogenesis to produce a mixture comprising
(a) volatile fatty acids and/or solvents such as short chain (C1 to C7) fatty acids, short chain (C1 to C7) alcohols, short chain (C1 to C7) ketones or any mixture of any two or more thereof, and
(b) undigested biomass, and

(2) subjecting at least a portion of the mixture to wet oxidation under conditions to reduce the volume of the undigested biomass while maintaining or increasing the concentration of the volatile fatty acids and/or solvents that are present, the wet oxidation conditions optionally comprising in one embodiment a residence time of less than about 120 minutes.

The following embodiments and preferences may relate alone or in any combination of any two or more to any of the above aspects.

In one embodiment the biomass comprises a hydrocarbon source. In various embodiments the biomass comprises a hydrocarbon source selected from the group comprising biological material, organic matter, plant matter, animal matter, waste material, organic waste material, plant waste material, animal waste material, dairy processing wastewater, abattoir wastewater, abattoir waste material, food processing wastewater, food processing waste material, wood pulp, lignocellulose pulp, pulp processing wastewater, pulp processing waste material, paper processing wastewater, paper processing waste material, municipal waste material, municipal wastewater, solids from municipal wastewater, lignocellulosic biomass, wastewater from lignocellulosic biomass processing, biosolid waste material from lignocellulosic biomass processing, or any combination of any two or more thereof.

In one embodiment the solids content of the biomass is at least about 0.5, 1, 5, 10, 15, 20, 25, 30, 35, 40, 45, 50, 55, 60, 65 or 70% by weight, and useful ranges may be selected between any of these values (for example, about 0.5 to about 5, about 0.5 to about 10, about 0.5 to about 15, about 0.5 to about 20, about 0.5 to about 25, about 0.5 to about 30, about 0.5 to about 35, about 0.5 to about 40, about 0.5 to about 45, about 0.5 to about 50, about 0.5 to about 55, about 0.5 to about 60, about 0.5 to about 65, or about 0.5 to about 70% by weight). At low solids concentrations, the biomass may also be useful to dilute other process streams, such as the mixture resulting from microbial digestion.

In one embodiment the biomass comprises one or more microorganisms. The microorganisms may be naturally present in the biomass or the biomass may be inoculated with one or more microorganisms. Suitable microorganisms are discussed below. In another embodiment the biomass substantially free of microorganisms, contains less than about 50,000 cfu/ml microorganisms or is substantially sterile.

In various embodiments, subjecting biomass to microbial digestion by contacting biomass with one or more microorganisms may be conducted in a biological reactor. The biological reactor may be an anaerobic tank or anaerobic digester, for example. The one or more microorganisms may be present in the biomass or may be added to the biomass.

The process of the invention includes applying conditions such that the microbial digestion of the organic biomass generates volatile fatty acids and/or solvents but minimises methanogenesis or other further digestion of the volatile fatty acids and/or solvents.

In one embodiment the microbial digestion conditions comprise a temperature of up to about 1, 5, 10, 15, 20, 25, 30, 25, 40, 45 or 50° C., and useful ranges may be selected between any of these values (for example, about 1 to about 10, about 1 to about 20, about 1 to 30, about 1 to about 40 and about 1 to about 50° C.).

In one embodiment the microbial digestion conditions comprise a pH of about 4, 4.5, 5, 5.5, 6 or 6.4, or a pH of about 7.3, 8, 8.5, 9, 9.5 or 10, and useful ranges may be selected between any of these values (for example, about 4 to about 6.4 or about 7.3 to about 10). In one embodiment the pH is preferably about pH 6. In another embodiment the pH is preferably about pH 8.

In one embodiment the microbial digestion conditions comprise a volatile suspended solids content of about 0.5, 1, 1.5, 2, 2.5, 3, 3.5, 4, 4.5, 5, 5.5, 6, 6.5, 7, 7.5, 8, 8.5, 9, 9.5 or 10 g/L, and useful ranges may be selected between any of these values (for example, about 0.5 to about 2, about 0.5 to about 3, about 0.5 to about 4 and about 0.5 to about 5).

In one embodiment the microbial digestion conditions comprise a digestion time of up to about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 days, and useful ranges may be selected between any of these values (for example, about 0.5 to about 20, about 5 to about 20, about 0.5 to about 15, about 0.5 to about 10, about 1 to about 10, about 2 to about 10, about 3 to about 10, about 4 to about 10, about 5 to about 10, about 6 to about 10, about 0.5 to about 8, about 1 to about 8, about 2 to about 8, about 3 to about 8, about 4 to about 8, about 5 to about 8, about 0.5 to about 6, about 1 to about 6, about 2 to about 6, about 3 to about 6, about 4 to about 6, and about 5 to about 6 days).

In one embodiment the microbial digestion is continued until the concentration of volatile fatty acids and/or solvents in the digestion medium reaches a maximum. As will be readily understood by a skilled reader, the concentration of volatile fatty acids and/or solvents can be monitored on a batch or continuous basis and microbial digestion halted on the basis of that monitoring. In one embodiment the concentration of volatile fatty acids and/or solvents is at least about 100, 105, 110, 115, 120, 125, 130, 135, 140, 145, 150, 160, 170, 180, 190, 200, 210, 220, 230, 240 or 250 mg/gVSS or more, and useful ranges may be selected between any of these values (for example, about 100 to about 250, about 100 to about 200 or about 150 to about 200). In one embodiment the concentration of acetic acid is at least about 40, 45, 50, 55, 60, 65, 70, 75, 80, 85, 90, 95, 100, 110, 120, 130, 140, 150, 160, 170, 180, 190 or 200 mg/gVSS, and useful ranges may be selected between any of these values (for example, about 40 to about 200, about 40 to about 100 or about 100 to about 150).

In one embodiment the method of the invention provides a gross yield of acetic acid, or of total volatile fatty acids (VFA), or both, that is at least about 10%, 20%, 30%, 40%, 50%, 60%, 70%, 80%, 90%, 100% or more greater than with wet oxidation of unfermented biosolids, and useful ranges may be selected between any of these values. In one embodiment the residence time in the wet oxidation stage is between about 30 minutes to about 4 hours.

For example, the method of the invention provides a gross yield of acetic acid that is at least about 10% to about 100% or more greater than with wet oxidation of unfermented biosolids. In a further embodiment, the method of the invention provides a gross yield of acetic acid over the first 1, 2, 3, 4, or 5 hours of oxidation that is at least about 10% to about 100% or more greater than with wet oxidation of unfermented biosolids.

In another example, the method of the invention provides a gross yield of volatile fatty acids that is at least about 10% to about 85% or more greater than with wet oxidation of unfermented biosolids. In a further embodiment, the method of the invention results in a gross yield of volatile fatty acids over the first 1, 2, 3, 4, or 5 hours of oxidation that is at least about 10% to about 85% or more greater than with wet oxidation of unfermented biosolids.

In one embodiment the method invention provides acetic acid purity, or provides volatile fatty acid purity, or both, that is at least about 10%, 20%, or 30% or more greater than with wet oxidation of unfermented by solids, and useful ranges may be selected between any of these values.

In one embodiment of the invention, the method of the invention provides an increase in the rate of production of acetic acid, or of total volatile fatty acids, or both, that is at least 10%, 20%, 30%, 40%, or 50% faster that the rate of production of acetic acid or total volatile fatty acids, or both from unfermented solids under similar wet oxidation conditions.

For example, the method of the invention provides an increase in the rate of production of acetic acid or total volatile fatty acids, or both that is at least 10%, 20%, 30%, 40% or 50% or more faster than the rate of production of acetic acid or total volatile fatty acids, or both from unfermented solids under similar wet oxidation conditions when the residence time in the wet oxidation stage is between 30 minutes and 4 hours. For example, the method of the invention results in an increase in the rate of production of acetic acid or of total volatile fatty acids, or both over the first 1, 2, 3, 4, or 5 hours of wet oxidation that is at least 10%, 20%, 30%, 40% or 50% or more faster than the rate of production of acetic acid or total volatile fatty acids, or both from unfermented solids under similar wet oxidation conditions.

In one embodiment the one or more microorganisms produce volatile fatty acids and/or solvents but minimise methanogenesis or minimise digestion of the volatile fatty acids and/or solvents.

In one embodiment the microbial digestion of the organic biomass is optimised using a combination of digestion conditions and one or more microorganisms to generate volatile fatty acids and/or solvents but minimise methanogenesis or minimise digestion of volatile fatty acids and/or solvents.

In various embodiments the microbial digestion conditions or the one or more microorganisms comprises one or more mixed cultures or one or more monocultures of bacteria or algae or a combination thereof. The culture comprises at least about 10³ cfu/ml, 10⁴ cfu/ml, 10⁵ cfu/ml or 10⁶ cfu/ml of the one or more microorganisms.

In one embodiment the culture is selected to improve the yield of volatile fatty acids and/or solvents. In one embodiment the culture is selected to reduce the production of methane.

In one embodiment the culture comprises one or more acidogenic microorganisms such as one or more acidogenic bacteria. Representative genera include but are not limited to Acetobacterium, Aeromonas, Clostridia, Klebsiella, Moorella and Ruminococcus, and any combination of any two or more thereof. Representative species include but are not limited to Acetobacterium spp., Aeromonas spp., Clostridia spp., Klebsiella spp., Moorella spp. and Ruminococcus spp., including but not limited to Acetobacterium woodii, Clostridium thermoaceticum, Clostridium thermolacticum, Clostridium ljungdahlii, Clostridium acetobutylicum, Clostridium formicaceticum, Clostridium glycolicum, Moorella thermoautotrophica, and Ruminococcus productus, and any combination of any two or more thereof. It should be understood that acidogenic microorganisms are naturally occurring in biomass such as municipal waste and many such microorganisms are reported in the literature and are suitable for use in the methods of the invention.

In one embodiment the culture comprises one or more acetogenic microorganisms such as one or more acetogenic bacteria. In this specification and claims, an acetogenic microorganism is a microorganism that is able to form acetate, irrespective of the mechanism of formation. Representative genera include Acetobacterium, Clostridium, Moorella and Ruminococcus, and any combination of any two or more thereof. Representative species include Acetobacterium woodii, Clostridium thermoaceticum, Clostridium thermolacticum, Clostridium ljungdahlii, Clostridium acetobutylicum, Clostridium formicaceticum, Clostridium glycolicum, Moorella thermoacetica, Moorella thermoautotrophica and Ruminococcus productus, and any combination of any two or more thereof. It should be understood that acetogenic microorganisms are naturally occurring in biomass such as municipal waste and many such microorganisms are reported in the literature and are suitable for use in the methods of the invention.

In one embodiment the culture comprises one or more acidogenic or acetogenic algae. Representative species include red algae.

In one embodiment the culture comprises less than about 10⁵ or 10⁶ cfu/ml of methanogenic microorganisms or the culture is substantially free of methanogenic microorganisms. In this specification and claims, a methanogenic microorganism is one that forms methane as a by-product of its metabolism, optionally one that preferentially forms methane as a by-product of its metabolism. Representative organisms include Methanobacteriaceae, Methanosaeta, and Methanosarcina.

In another embodiment the microbial digestion conditions are substantially free of hydrogen gas (H₂). In this embodiment where an anaerobic bioreactor is used, hydrogen gas is removed from the headspace of the bioreactor. Removing hydrogen removes nutrient source required by methanogenic bacteria to produce methane.

In one embodiment the microbial digestion conditions are substantially free of one or more biomass components or contaminants that reduce the concentration of volatile free fatty acids and/or solvents.

In one embodiment, one or more additives are added to the biomass before, during or after microbial digestion. The one or more additives may comprise any one or more of additional biomass, one or more microorganisms, one or more methanogenesis inhibitors and/or acid or base to adjust pH, for example, or any combination of any two or more thereof.

In one embodiment the microbial digestion conditions further comprise a methanogenesis inhibitor. Many such inhibitors are known in the art. In one embodiment the methanogenesis inhibitor is selected from ethylene, bromoalkanes including bromoethane, sulfonic acid, nitrate, acetylene and low levels of oxygen, and any combination of any two or more thereof.

In one embodiment the solids content of the mixture resulting from microbial digestion is, or is diluted or dewatered to, about 0.5, 1, 1.5, 2, 2.5, 3, 3.5, 4, 4.5, 5, 5.5, 6, 6.5, 7, 7.5, 8, 8.5, 9, 9.5 or 10% by weight, and useful ranges may be selected between any of these values (for example, about 0.5 to about 6, about 0.5 to about 7, about 0.5 to about 8, about 0.5 to about 9, about 0.5 to about 10, about 2 to about 6, about 2 to about 7, about 2 to about 8, about 2 to about 9, about 2 to about 10, about 3 to about 6, about 3 to about 7, about 3 to about 8, about 3 to about 9 or about 3 to about 10% by weight).

In one embodiment the solids content of the mixture resulting from microbial digestion is adjusted before being subjected to wet oxidation. In one embodiment the mixture resulting from microbial digestion is diluted. In one embodiment the mixture resulting from microbial digestion is de-watered.

In one embodiment the wet oxidation conditions comprise a temperature of up to the critical point of water, including a temperature of at least about 100, 110, 120, 130, 140, 150, 160, 170, 180, 190, 200, 210, 220, 230, 240, 250, 260, 270, 280, 290, 300, 310, 320, 330, 340, 350, 360, 370 or 374° C., and useful ranges may be selected between any of these values (for example, about 100 to about 374, about 100 to about 320, about 125 to 320, about 165 to about 265 and about 165 to about 220° C.).

In one embodiment the wet oxidation conditions comprise an oxidant, optionally selected from air, purified air, oxygen, or peroxide. The concentration of the oxidant is dependent on the solids content of the mixture entering the wet oxidation stage. On a chemical oxygen demand (COD) basis, the concentration of the oxidant may beneficially be below, at or above the stoichiometric ratio for complete oxidation of the organic material in the mixture entering the wet oxidation stage. In one embodiment the oxidant concentration is at least about 0.5. 0.75, 1, 1.5 or 2 times the stoichiometric amount required for complete oxidation of the organic material in the mixture entering the wet oxidation stage. In one embodiment the wet oxidation conditions comprise an oxygen concentration of at least about 10, 15, 20, 25 or 30 bar oxygen, and useful ranges may be selected between any of these values (for example, about 10 to about 30 bar oxygen).

In one embodiment the wet oxidation conditions comprise a residence time of up to about 5, 10, 15, 20, 25, 30, 35, 40, 45, 50, 55, 60, 65, 70, 75, 80, 85, 90, 95, 100, 105, 110, 115, 120, 150 or 180 minutes, or about 0.5, 1, 1.5, 2, 2.5, 3, 3.5, 4, 4.5, 5, 5.5, 6, 6.5, 7, 7.5 or 8 hours, and useful ranges may be selected between any of these values (for example, about 5 to about 180 minutes, about 5 to about 120 minutes, about 15 to about 120 minutes, about 5 to about 60 and about 15 to about 60 minutes, about 0.5 to about 3 hours, about 0.5 to about 4 hours, about 0.5 to about 5 hours, about 0.5 to about 6 hours, about 0.5 to about 7 hours, and about 0.5 to about 8 hours).

In one embodiment subjecting the mixture to wet oxidation increases the total accumulated mass of carbon in the form of volatile fatty acids and/or solvents.

In one embodiment the wet oxidation conditions produce additional volatile fatty acids while minimising the oxidation of volatile fatty acids and solvents to CO₂.

In one embodiment the wet oxidation conditions reduce the volume of biosolids while avoiding a net reduction in the concentration of volatile fatty acids and/or solvents compared to the concentration of volatile fatty acids and/or solvents present before wet oxidation. In one embodiment the wet oxidation conditions maximise the destruction of biosolids without mass destruction of the volatile fatty acids and/or solvents.

In one embodiment the wet oxidation conditions reduce the volume of biosolids, that is, reduce total suspended solids (TSS), by at least about 60, 70, 80, 90, 95 or 99%, and useful ranges may be selected between any of these values (for example, about 60 to 99, about 70 to 99, about 80 to 99 or about 90 to 99%).

In one embodiment, one or more additives are added to the mixture before, during or after wet oxidation. The one or more additives may comprise any one or more of additional biomass and/or one or more oxidants, for example, or a combination thereof.

In one embodiment the process includes pre-treatment of the biomass by wet oxidation, preferably short duration wet oxidation, to reduce the viscosity of the biomass or improve the solubilisation of the biomass or both.

In one embodiment the process includes sterilisation of the biomass before microbial digestion followed by inoculation with unsterilised biomass, a mixed culture or one or more monocultures, or any combination of any two or more thereof. Suitable organisms are discussed above. In one embodiment the sterilisation comprises wet oxidation or thermal hydrolysis.

In one embodiment the process further comprises separating at least one of the volatile fatty acids or solvents from the mixture following wet oxidation.

In another embodiment the process further comprises separating ammonium from the mixture following wet oxidation.

In yet another embodiment the process further comprises separating a precipitated phosphorus-containing compound from the mixture following wet oxidation.

In one embodiment at least one of the separated volatile fatty acids or solvents is used as a feedstock for microbial digestion of biomass.

In one embodiment the separated ammonium is used as a buffer for pH control of microbial digestion conditions.

In one embodiment the separated ammonium and phosphorous containing-compound are processed into fertiliser.

In one embodiment at least one of the separated volatile fatty acids or solvents is processed into a fuel or fuel precursor. Accordingly, a further aspect of the invention relates to a process of producing a fuel or fuel precursor, the process comprising processing at least one of the separated volatile fatty acids or solvents produced by a method of the above aspects into a fuel or fuel precursor. In one embodiment the fuel or fuel precursor comprises alcohol.

In one embodiment the process results in conversion of at least about 30, 40, 50, 60, 70, 80 or 90% of organic nitrogen in the biomass to ammonium, and useful ranges may be selected between any of these values (for example, about 30 to about 90%).

In one embodiment an amount of liquid from wet oxidation is added to an amount of the mixture before the mixture is subjected to wet oxidation. In one embodiment the amount of liquid is selected to dilute the mixture to a solids content of about 0.5 to about 10% by weight, as discussed above.

This recycle step can be employed in a continuous process or in a batch process. In a batch process, liquid from wet oxidation of a first batch of mixture is added to a second batch of mixture. In one embodiment the liquid is processed, such as by filtration or settling to reduce or remove ash or metals, including heavy metals, or to reduce the content of both ash and metals.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE FIGURES

FIG. 1 is a flowchart depicting the method of the invention.

FIGS. 2 to 5 are graphs showing acetic acid yield (mg acetic acid per g of volatile suspended solids [VSS] of biomass feedstock) of a process of the invention (W) after a residence time in the fermentation stage of 6 days (FIGS. 2 and 4) or 7 days (FIGS. 3 and 5) at pH 6 (FIGS. 2 and 3) or pH 8 (FIGS. 4 and 5), compared to initial biomass feedstock control samples (Feed), biomass samples that were subjected to fermentation only (U), and biomass samples that were subjected to wet oxidation only (WO).

FIG. 6 is a graph showing volatile suspended solids (VSS) destruction of biomass treated according to Example 2.

FIGS. 7 and 8 are graphs showing the change in soluble organics between process stages, as measured by acetic acid and total VFA (FIG. 7) and soluble COD and dissolved organic carbon (DOC) (FIG. 8).

FIG. 9 is two graphs showing gross and net acetic acid yields in batch wet oxidation.

FIG. 10 is two graphs showing gross and net total VFA yields in batch wet oxidation.

FIG. 11 is two graphs showing the purity of acetic acid and total VFA across time course of batch reaction.

### DETAILED DESCRIPTION

The present inventors have determined that a combination of microbial digestion (fermentation), preferably anaerobic microbial digestion and wet oxidation provides an improved ability to treat biomass such as municipal wastes to generate volatile fatty acids, acetone and/or short chain alcohols and reduce the volume of residual biosolids. When the feedstock to the process is a waste stream such as biosolids from municipal waste water the destruction of the biomass may greatly reduce the demands for land-filling and incineration. In various embodiments, the process of the invention allows separation of carbon, nitrogen and phosphorous components in readily processable forms.

In general terms, the present invention provides a combined microbial digestion—wet oxidation process comprising

(1) microbial digestion, preferably anaerobic microbial digestion to produce better yield and a greater variety of volatile fatty acids and solvents per quantity of biomass digested than wet air oxidation,

(2) wet oxidation to produce more volatile fatty acids and destroy residual biomass,

(3) wet oxidation conditions adapted to retain a useful concentration of volatile fatty acids and/or solvents produced in the microbial digestion stage while at the same time generating additional volatile fatty acids and/or solvents from the residual biomass,

(4) production of an accumulated yield of usable carbon in the form of volatile fatty acids and solvents, greater than possible through wet oxidation alone while retaining the capability to destroy the vast majority of the biosolids.

The process retains the wet oxidation advantages of the resulting mixture being in a form where it is less difficult to separate the volatile fatty acids and/or solvents from the processed waste stream, while avoiding methanogenesis and loss of organic carbon by oxidation to CO₂. The process also has the added advantage typical of wet oxidation processes of destruction of pathogens in waste water.

A major benefit of the two-stage process described herein is that the conversion of biomass carbon into VFA/solvent in the microbial process lowers the oxygen requirement within the wet oxidation stage. This outcome has the potential for major savings in operational and capital infrastructure costs.

As a result, the overall process targets the enhancement of product yield. Solids destruction rate and extent is significantly enhanced over stand-alone biochemical fermentation, whilst VFA/solvent production is enhanced over a standalone wet oxidation process, with the additional benefit of reduced oxidation costs for the wet oxidation process step.

This enhanced conversion of organic biomass into VFA and/or solvent molecules, provides an end-product that is suitable for multiple downstream uses, biotechnological or otherwise.

It is noted that there is potentially an optima of conversion for each step of the process, requiring a balance of

(1) ensuring that there is sufficient organic biomass present within the fermentation stage effluent to enable autothermal operation of the wet oxidation process

(2) biochemical conversion extent (effected by time and impacting on capital and operating costs, but lowering oxidation costs of subsequent wet oxidation step)

(3) rapidity of destruction of the wet oxidation process, (the cost being lower product yield).

The yield and selection of products can be optimised by an additional wet oxidation rapid pre-treatment that both solubilised the mixture and sterilises it permitting use of pure culture(s) fermentation to target specific products and/or yield.

Pure culture fermentation allows fermentation for targeted product suites including VFAs, hydrogen, or solvents.

In addition to the improved yields and range of small carbon based molecules, the process can also convert a large proportion of organic nitrogen in the waste water to ammonium providing options for physical and chemical separation of a large proportion of the nitrogen from the carbon based products and the mostly mineral residue. Under wet oxidation conditions up to 90% of solid nitrogen is solubilised with the final liquor having approximately 75% of the nitrogen existing as ammonium.

The wet oxidation stage of the process also results in reduced concentrations of phosphorous in the liquid phase indicating precipitation again providing options for chemical and physical separation of this component.

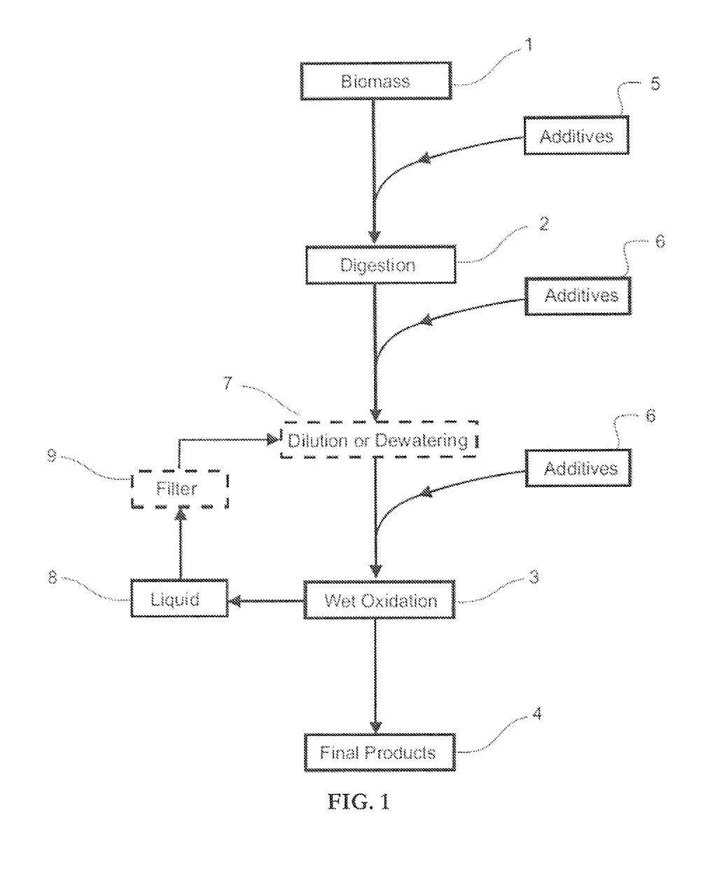

Referring generally to FIG. 1, a method of the invention comprises subjecting biomass (1) to microbial digestion (2) under conditions to balance the factors described above. Suitable biomass (1) and conditions and apparatus for microbial digestion (2) are described above and below. The second stage, wet oxidation (3), is conducted under conditions to balance the factors described above and suitable conditions and apparatus for wet oxidation (3) are described above and below. The final products (4) produced comprise volatile fatty acids and/or solvents and, optionally, useful forms of nitrogen and phosphorous, as described herein. Additives (5, 6) may be added to the reaction mixture of either stage, as described herein. Additives (5) at the microbial digestion stage (2) may comprise any one or more of additional biomass, one or more microorganisms, one or more methanogenesis inhibitors and/or acid or base to adjust pH, for example, or any combination of any two or more thereof. Additives (6) at the wet oxidation stage (3) may comprise any one or more of additional biomass and/or one or more oxidants, for example, or a combination thereof.

As discussed above, the solids content of the mixture resulting from microbial digestion may be diluted or dewatered (7) to about 0.5 to 10% by weight. Dilution can be achieved by, for example, addition of water, dilute biomass (as described above) or liquid obtained from a wet oxidation process. De-watering can be achieved by, for example, dehydration or filtration using known techniques.

Liquid (8) can be obtained from the wet oxidation stage and recycled to dilute an amount of digested biomass mixture entering a wet oxidation stage. In a batch process, the liquid (8) for recycle would be obtained from an earlier batch. In a continuous process, the liquid (8) for recycle would be drawn off the wet oxidation reactor or from a fluid previously extracted from the reactor and recycled to digested biomass mixture entering the wet oxidation stage. Optionally the liquid (8) may be processed, such as filtered or settled (9), to reduce the content of ash or metals, including heavy metals, or to reduce the content of both ash and metals.

The term “acidogenesis” refers to the second stage (following hydrolysis) in the four stages of anaerobic digestion: A biological reaction where simple monomers are converted into volatile fatty acids.

The term “acetogenesis” refers to a process through which acetate is produced by anaerobic microorganisms from a variety of energy and carbon sources.

The term “comprising” as used in this specification means “consisting at least in part of”. When interpreting statements in this specification which include that term, the features, prefaced by that term in each statement or claim, all need to be present but other features can also be present. Related terms such as “comprise” and “comprised” are to be interpreted in the same manner.

The phrase “mass destruction” in relation to a stated element, compound or substance means a net reduction in the concentration of the stated element, compound or substance (for example, volatile fatty acids and/or solvents) compared to the concentration of the stated element, compound or substance that is present immediately preceding treatment by wet oxidation.

The term “methanogenesis” refers to a biological reaction where acetates or other small organic compounds are converted by microorganisms including bacterium and Archea into methane.

The term “solvents” means non-aromatic alcohols or ketones with a linear or branched carbon chain of one to seven carbon atoms, including but not limited to alcohols such as methanol, ethanol, propanol, isopropanol, butanol, sec-butanol, isobutanol, tert-butanol, pentanol, hexanol, and heptanol, and ketones such as propanone (acetone).

The phrase “substantially free” means that a composition contains very little of the stated element, compound, substance or organism as a proportion of total weight, for example less than about 1.0, 0.75, 0.5, 0.25, 0.2, 0.175, 0.15, 0.125, 0.1, 0.075, 0.05, 0.025 or 0.01% by weight of the stated element, compound, substance or organism, and useful ranges may be selected between any of these values (for example, about 0.01 to about 1.0, about 0.01 to about 0.2, about 0.01 to about 0.175, about 0.01 to about 0.15, about 0.01 to about 0.125, about 0.01 to about 0.1, or about 0.01 to about 0.075%).

The phrase “volatile fatty acids” means fatty acids with a linear or branched carbon chain of one to seven carbon atoms (C1 to C7), optionally substituted by —COOH or —OH, including but not limited to methanoic (formic) acid, ethanoic (acetic) acid, propanoic (propionic) acid, butanoic (butyric) acid, pentanoic (valeric) acid, hexanoic (caproic) acid, heptanoic (enanthic) acid, branched variants thereof (including, for example, iso-butyric acid, n-butyric acid, and butyric lactic acid), and esters and salts thereof.

### 2. Microbial Digestion (Fermentation)

The described process may be readily adapted to continuous, batch and semi-continuous processes by techniques that are well known in the relevant arts.

In the microbial digestion stage, polymers associated with the biomass are hydrolysed to substrates which can be utilised by microorganisms as an energy and growth source, under anaerobic conditions. The end-products of the fermentation stage are short chain fatty acids (VFA) and solvents. Examples of these include, but are not limited to, acetic, propionic, butyric, formic and lactic acids (VFA), and methanol, ethanol, acetone, butanol, propanol (solvents). The biochemical reactions occurring are typically termed hydrolysis, acidogenesis and solventogenesis.

Anaerobic fermentation can be typified by the following general steps in degradation of polymers such as proteins and carbohydrates.
(1) Hydrolysis: breakdown of polymers into smaller fractions (monomers, dimers etc) suitable for uptake by microorganisms.
(2) Acidogenesis: conversion of these hydrolysis products into VFA and solvents.
(3) Acetogenesis: conversion of longer chain VFA and solvents into mainly acetic acid, carbon dioxide and hydrogen.
(4) Methanogenesis: conversion of acetates into methane and carbon dioxide, while requiring a hydrogen source.

The aim of the fermentation unit process in this invention is to optimise yield (conversion efficiency) and product range, via biochemical conversion. The advantages of using biological fermentation are that the product range and yield are enhanced over use of wet oxidation alone.

This stage of the process can be used to produce acetic, propionic, butyric, valeric, caproic and heptanoic acids to percentage levels of the total VFA production from the anaerobic fermentation.

### Batch Fermentation

Small, medium and large scale batch fermentation may be conducted using appropriate reactor vessels such as ponds or tanks. At small scale, glass reactors of 5 L total volume (2-4 L working volume) may be used. At medium and large scale, tanks or ponds may be more suitable.

Biomass is initially introduced into the vessel to obtain a desired initial volatile suspended solids (VSS) concentration, typically in the range of 2 to 4% by weight, particularly at small scale.

Useful biomass includes but is not limited to a hydrocarbon source selected from the group comprising biological material, organic matter, plant matter, animal matter, waste material, organic waste material, plant waste material, animal waste material, dairy processing wastewater, abattoir wastewater, abattoir waste material, food processing wastewater, food processing waste material, wood pulp, lignocellulose pulp, pulp processing wastewater, pulp processing waste material, paper processing wastewater, paper processing waste material, municipal waste material, municipal wastewater, solids from municipal wastewater, lignocellulosic biomass, wastewater from lignocellulosic biomass processing, biosolid waste material from lignocellulosic biomass processing, or any combination of any two or more thereof.

Useful wastewater solids can include primary, secondary or tertiary sludges or biosolids from biological wastewater treatment plants or combined biological and chemical sludges from wastewater treatment plants. Wastewater treatment plants includes those treating domestic wastewater or industrial wastewaters such as dairy processing wastewater, lignocellulosic processing wastewater, pulp and paper wastewater, and food processing wastewater.

Useful plant matter can include agricultural, food or energy crop residues such as crop straws or bagasse.

Useful animal waste material can include agricultural residues such as piggery or dairy effluents.

Vessels are desirably continuously stirred or agitated using known apparatus. Mixing may be mechanical and/or hydraulic. For hydraulic mixing, this may be provided by gas or liquid recirculation. Example reactor configurations for mixing include internally stirred impellor, gas lift, or bubble column type reactors.

Temperature is controlled in the range of about 25 to about 70° C., with the actual temperature being dictated by the nature of the biomass and microorganisms present. For example, a temperature of about 30 to about 45° C., preferably 36° C., is suitable for medium scale processing of municipal wastewater. Temperature control is achieved through use of any suitable means of direct or indirect heating such as heating of input feed, water jackets or recirculating water loops in the reactors. Elevating temperature into the thermophilic range has some theoretical thermodynamic advantages for acetate production. Further, the stability of methanogenesis may be compromised at elevated temperatures, including for example, a temperature of about 50 to about 60° C.

Micro-aerobic or anaerobic conditions may be maintained through use of sealed vessels, including hermetically sealed vessels and appropriate arrangements allowing for the removal of gas—such as water traps. Headspace gases may be removed using known apparatus. Partial pressures of reactive gases such as CO₂ and H₂ may impact on the productivity of system (Kraemer and Bagley, 2007). These levels are manipulated by the reactor operational parameters, including system pressure, presence of sparging, hydrodynamic shear etc.

pH is recorded using known apparatus and controlled via alkali addition (NaOH, for example). Regular pH adjustment may be required for fermentation, including adjustment about every one, every two or every three days. In one embodiment the microbial digestion conditions are adjusted to maintain a pH of about 4, 4.5, 5, 5.5, 6 or 6.4, or a pH of about 7.3, 7.5, 8, 8.5, 9, 9.5 or 10, and useful ranges may be selected between any of these values (for example, about 4 to about 6.4 or about 7.3 to about 10). In one embodiment the pH is preferably pH 8. Maintaining the pH outside the generally considered optima for methanogenesis of pH 6.5 to 7.2 (Appels et al., 2008) is preferred.

If biomass is initially sterilised or if otherwise desired, one or more microorganisms may be added to the biomass at 0.5 g/L by VSS of a culture broth. The one or more microorganisms may comprise one or more monocultures, one or more mixed cultures or an unsterilized amount of biomass material comprising one or more microorganisms, as described above. Initial sterilisation of biomass may be useful to inactivate undesirable bacteria that are present, such as methanogenic bacteria and bacteria that produce hydrogen gas including Clostridia spp.

A target residence time for microbial digestion is about 0.5 to about 20 days, including about 0.5 to about 10, about 0.5 to about 7, or about 6 to about 7 days. To optimise reduction of methanogenesis, a residence time of about 0.5 to about 10, about 0.5 to about 7, or about 6 to about 7 days is preferred.

When required, chemical inhibitors of methanogenesis such as ethylene, bromoalkanes including bromoethane, sulfonic acid, and low levels of oxygen (Wang and Wan, 2009) may be employed.

### Continuous Fermentation

Continuous fermentation may generally be conducted with the same apparatus and process conditions as batch fermentation described above, with automation of pH control to provide continuous controlled addition of alkali and with batch, fed-batch, semi-continuous or continuous addition and withdrawal of solids to provide a residence time of about 0.5 to about 20 days, including about 0.5 to about 10, about 0.5 to about 7, or about 6 to about 7 days. VSS concentration of subsequent feed material (at about 40 to about 50 g/L, for example) may be higher than the initial fermentation starting material at day 0 (at about 30 g/L, for example).

### Process Conditions

One aim of microbial digestion is to maximise VFA production, including acetic acid, in part through minimising competitive end-products. Chief amongst these competitors is methane Minimising carbon loss to methane can be managed through a combination of
(1) reduced residence time,
(2) controlled pH,
(3) inactivating methanogens, and/or
(4) use of methanogenesis inhibitors.

A related aim of microbial digestion is to improve process efficiency, for example by enhancing total yield or purity of VFA products, or by minimising time or energy requirements. Applicants believe, without wishing to be bound by any theory, that microbial digestion increases the production of desirable chemical precursors that are more readily converted into targeted VFA products including acetic acid. Again, without wishing to be bound by any theory, Applicants attribute the increased rates of production and process efficiency associated with embodiments of the present invention and increased total yield in acetic acid and VFA, particularly over the first 30 minutes to 4 to 5 hours of wet oxidation, at least in part to the production of desirable precursor molecules by microbial digestion.

### 3. Wet Oxidation

Wet oxidation has been reviewed by Bhargava et al., 2006 and Mishra et al., 1995, incorporated herein by reference. In the wet oxidation stage, the mixture discharged from the fermentation process is exposed to high temperature and/or pressure, under an oxidative environment, maintained by the addition of (for example) air, oxygen or hydrogen peroxide. High levels of organic biomass destruction are possible, ranging from 60 to 90 or 60 to 99% depending on process conditions. Biomass destruction is otherwise referred to herein as a reduction in the volume of biosolids and is monitored through measurement of total suspended solids (TSS). If the prime purpose of wet oxidation is destruction of biosolids, the organic biomass may be oxidised to carbon dioxide and vented to the atmosphere but reaction conditions can be manipulated to prevent all the biosolids and incoming volatile fatty acids and solvents being converted to carbon dioxide and instead converting at least a portion of the biosolids to small carbon molecules, primarily acetate and other volatile fatty acids.

### Batch and Continuous Wet Oxidation Apparatus

Small, medium or large scale wet oxidation may be conducted in a batch, semi-batch or continuous process in a suitable pressure vessel, including bench top reactors (such as from Parr Instrument Company, Model 4540, total volume: 600 ml) through to sub-surface wells. A typical processing volume is dictated by the choice of reactor vessel.

Biomass solids from the digestion stage may be added at a consistency of about 3 to about 6% by weight total suspended solids (TSS), with optional blending to ensure greater homogeneity and to improve handling characteristics for transfer to the pressure vessel.

Typical wet oxidation conditions are described above, with one example being an oxygen overpressure of about 20 bar, an operating temperature of about 220° C., and a residence time of about two hours with optional mechanical and/or hydraulic mixing.

In a batch process, all components are added to the pressure vessel simultaneously, with the vessel then being sealed and heating initiated.

In a semi-batch process, biomass is added batch-wise to the vessel, whilst other components such as the oxidant (such as air, purified air, oxygen, or peroxide such as hydrogen peroxide, for example) are added continuously to the reactor during the heating and reaction phases.

In a continuous process, biomass and oxidant are continuously added.

At large scale, mixing may be through gas or liquid recirculation (or both), or by transfer of gas-liquid solid matrix from inlet to outlet.

Heating may be achieved through heat exchangers passing thermal energy from hot outgoing process fluid to the colder incoming material.

In one embodiment the wet oxidation conditions comprise a temperature of at least about 100, 110, 120, 130, 140, 150, 160, 170, 180, 190, 200, 210, 220, 230, 240, 250, 260, 270, 280, 290, 300, 310, 320 or 330° C. or more, and useful ranges may be selected between any of these values (for example, about 100 to about 320, about 125 to 320, about 165 to about 265 and about 165 to about 220° C.).

Pressure is temperature dependant due to the vapour pressure of water at a given temperature. Pressures may range from 0.5-20 Mpa at a temperature of 165-320° C.

In one embodiment the wet oxidation conditions comprise a residence time of up to about 5, 10, 15, 20, 25, 30, 35, 40, 45, 50, 55, 60, 65, 70, 75, 80, 85, 90, 95, 100, 105, 110, 115, 120, 150 or 180 minutes, or about 0.5, 1, 1.5, 2, 2.5, 3, 3.5, 4, 4.5, 5, 5.5, 6, 6.5, 7, 7.5 or 8 hours, and useful ranges may be selected between any of these values (for example, about 5 to about 180 minutes, about 5 to about 120 minutes, about 15 to about 120 minutes, about 5 to about 60 and about 15 to about 60 minutes, about 0.5 to about 3 hours, about 0.5 to about 4 hours, about 0.5 to about 5 hours, about 0.5 to about 6 hours, about 0.5 to about 7 hours, and about 0.5 to about 8 hours).

In one embodiment the wet oxidation conditions reduce the volume of biosolids, that is, reduce total suspended solids (TSS), by at least about 60, 70, 80, 90, 95 or 99%, and useful ranges may be selected between any of these values (for example, about 60 to 99, about 70 to 99, about 80 to 99 or about 90 to 99%).

In various embodiments catalysts may be added. Common catalysts include iron, copper and a number of other transition metals, and activated carbon complexes.

Various aspects of the invention will now be illustrated in non-limiting ways by reference to the following examples.

### EXAMPLES

### Example 1

This example demonstrates the operation of the method described herein.

### Fermentation Method

Glass reactors of 5 L total volume (2-4 L working volume) were used. These were continuously stirred with paddle stirrers. Temperature was controlled to 36° C., through recirculating water loops in the reactors. The fermenters were sealed from the laboratory environment via a water trap arrangement, thus allowing anaerobic conditions to prevail.

pH was recorded and automatically controlled via alkali addition (NaOH) at pH 6 or pH 8.

Biomass in the form of municipal biosolids was charged into the reactors at the initiation of the experiment. Volatile suspended solids (VSS) concentration of fermentation starting material (day 0) was 30 g/L, whilst subsequent feed-in concentrations were 40 and 50 g/L.

Fermentations were semi-continuous, with batch withdrawal and feeding every 2 or 3 days, giving residence times of 6 or 7 days.

### Wet Oxidation Method

A 200 ml sample of fermentation biomass was subjected to a batch wet oxidation. This was conducted in a Parr high pressure reactor (Parr Instrument Company, Model 4540, total volume: 600 ml) equipped with a stirrer and heating jacket. An oxygen overpressure of 20 bar was added (BOC NZ Ltd—zero grade), and the reactor was heated to 220° C. for a total reaction time of two hours (from initial heat-up), and stirring at 400-500 rpm.

### Analysis

The water quality parameters, total suspended solids (TSS), volatile suspended solids (VSS), dissolved organic carbon (DOC), total chemical oxygen demand (COD), soluble chemical oxygen demand (SCOD) and particulate chemical oxygen demand (PCOD) were measured following standard analytical procedures (APHA, 1998).

The volatile fatty acids (VFA) were determined by a method involving pH correction with formic acid, followed by capillary gas chromatography with flame ionisation detection (GC-FID). The column used was a 30 m×0.53 μm ID Nukol™ ramped from 40 to 150 W C. Butan-1-ol solution was used as the internal standard. The total residual organic carbon concentration (TOC) in filtered samples was also measured with a TOC analyser (Elementar High TOC II).

Nitrite (NO 2-N), nitrate (NO 3-N), total Kjeldahl nitrogen (TKN) and dissolved reactive phosphorus (DRP; as PO 4-P) species were determined according to standard methods (APHA, 1998).

### Results

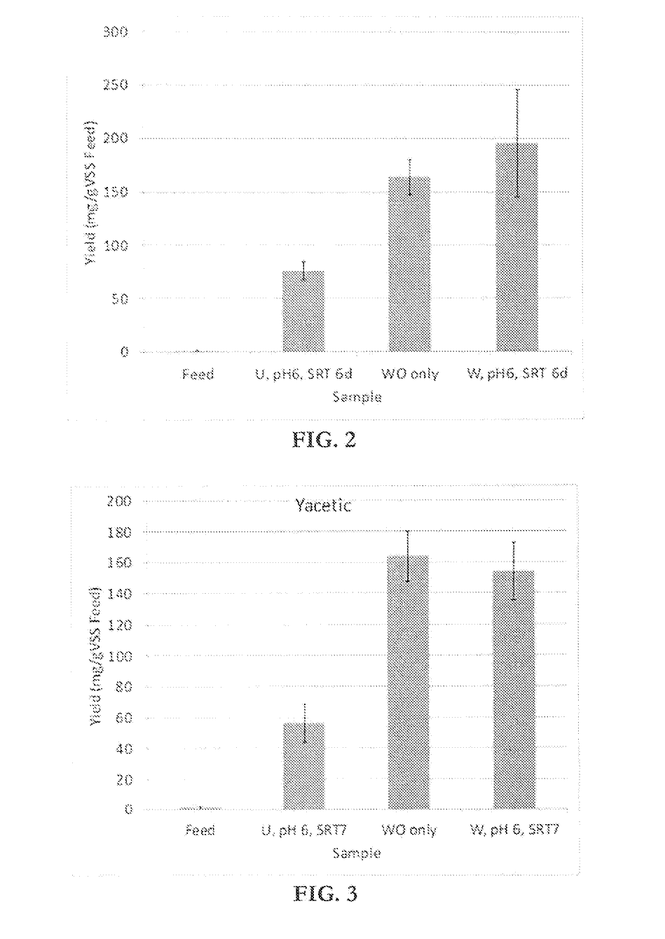

Acetic acid yields are shown in FIGS. 2 to 5.

### Example 2

This example demonstrates that the method of the invention allows the intensity (temperature/time) of the wet oxidation process to be reduced, while maintaining an acceptably high VSS destruction level.

Waste activated sludge from the Rotorua District Council waste water treatment plant was batch fermented under acidogenic conditions for 15 days. The pH was maintained at above or below the 6.8 to 7.2 band that is optimal for methanogenesis. Four reactors were operated under the following conditions of 36° C., pH 6 (Reactor 9/10) or 8 (R11/12), VSS of waste sludge 3 g/l and VSS of AD inoculum 0.5 g/l. This inocula was sourced from a previous batch fermentation of similar biomass material.

Samples were removed after specific time periods and underwent wet oxidation as received (i.e. unprocessed), or by fractionating samples into the liquid and solid phases. The liquid sample volume was measured and made up to 200 g with distilled water, while the solids were washed twice using distilled water and resuspended in distilled water to 200 g (at the liquor pH). Unprocessed and fractionated samples then underwent we oxidation under the following conditions: loading: 200 g of partially fermented sludge, temperature: 220° C., reaction time: 2 hours total (heating+reaction time), oxidant concentration: 20 bar oxygen, and stirrer speed: 350 rpm.

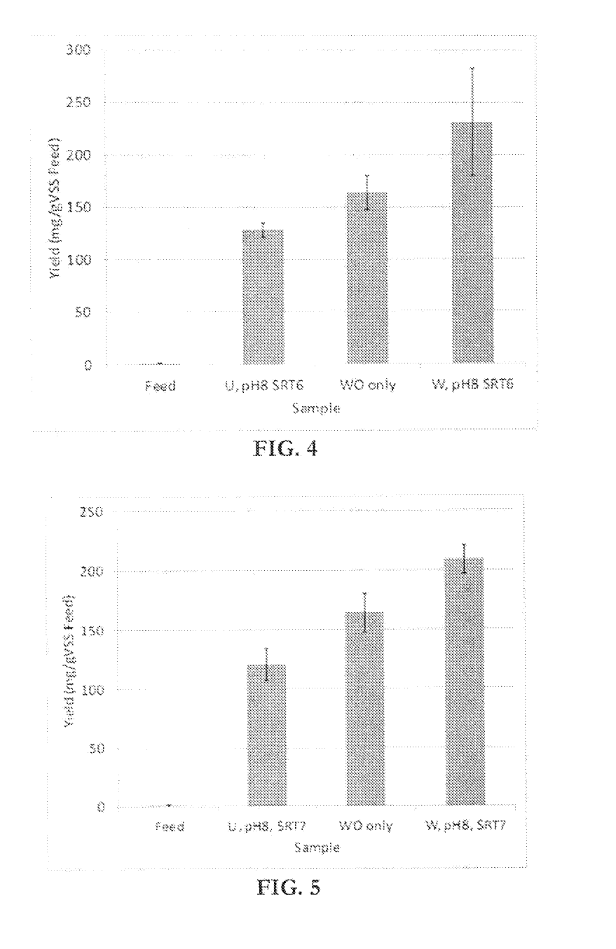

The results are shown in FIG. 6. The increased destruction compared to a single stage wet oxidation was approximately 3-4% and was attributable to the reduced VSS load encountered during the wet oxidation stage of the hybrid process. This finding presents an opportunity to reduce the intensity (temp/time) of the wet oxidation process, while maintaining an acceptably high VSS destruction level.

### Example 3

This example demonstrates that dilution of digested biomass with liquid from a separate wet oxidation reaction produces soluble organic concentrations (as measured by soluble TOC) that are higher than with a process using a single wet oxidation stage.

### Method

Biosolids was sourced from a full-scale wastewater treatment plant running an activated sludge process and was obtained from the thickened solids transported offsite after activated sludge treatment of municipal wastewater.

An experiment consisting of three batch wet oxidation reactions was conducted on the sample. Each wet oxidation was conducted in a Parr reactor (Parr Instruments Co, USA), with 600 mL total volume. 200 mL samples were processed, with starting oxygen partial pressure of 20 bar. Each reaction was raised from ambient temperatures to 220° C., with a total reaction time of 120 minutes. 200 mL fresh biosolids at 3% solids by weight (S0) was processed in the first wet oxidation stage. 192 mL wet oxidised biomass was obtained from stage 1 (S1) and 100 mL of that was added to the second wet oxidation stage, along with 100 mL of fresh biosolids at 6% solids by weight (S4). 192 mL wet oxidised biomass was obtained from stage 2 (S2) and 100 mL of that was added to the third wet oxidation stage, along with 100 mL of fresh biosolids at 6% solids by weight (S4). 192 mL wet oxidised biomass was obtained from stage 3 (S3)

Standard analytical procedures (APHA, 1998) were conducted for total and volatile suspended solids (TSS and VSS), ash, soluble total organic carbon (sol TOC) total chemical oxygen demand (totCOD) and selected organic acids and alcohols.

### Results

The results in Table 1 below demonstrate that dilution with wet oxidation liquor produces soluble organic concentrations (as measured by soluble TOC) which are higher than for a single stage wet oxidation. Further, acetic acid also increased in concentration across the stages.

TABLE 1
Results from multi-stage batch wet oxidation (mg/L)

| | | | | sol | | Acetic | Propionic | |
|---|---|---|---|---|---|---|---|---|
| Sample ID | TSS | VSS | Ash | TOC | TotCOD | Acid | Acid | Methanol |
| S1 | 3466 | 855 | 2611 | 4450 | 11780 | 3877 | 116 | 0 |
| S2 | 4616 | 773 | 3843 | 6031 | 14320 | 5170 | 124 | 42 |
| S3 | 5269 | 707 | 4562 | 6533 | 13420 | 5782 | 115 | 44 |
| S0 (~3% solids) | 24657 | 21565 | 3092 | 615 | 38800 | 0 | 0 | 0 |
| S4 (~6% solids) | 39514 | 33711 | 5803 | 1864 | 51695 | 0 | 0 | 0 |

### Example 4

This example demonstrates the operation of the method described herein.

### Fermentation Method

A 2000 L total volume (1000 L working volume) pilot plant fermentation reactor was used. This was continuously stirred, mechanically, and the temperature maintained at 45° C. via a water jacket. Anaerobic conditions were maintained throughout and nitrogen was added to the headspace as required during sludge discharge to maintain a positive pressure.

The pH was recorded and automatically controlled via acid (H₂SO₄) or alkali (NaOH) addition to a setpoint of pH 6.2.

Municipal biosolids were automatically fed, three times daily, into the fermentation reactor and fermented material was discharged. Volatile suspended solids (VSS) concentration of feed substrate was on average 42,500 mg/l. The feed rate was set to ensure there was a 4 day solids retention time in the fermentation reactor.

### Wet Oxidation Method

A 200 L total reactor volume (80 L working volume) pilot plant wet oxidation pressure vessel was used. This was mixed via liquid recirculation and gas recirculation pumps. The pressure vessel was raised to a working temperature of 220° C. using water.

Fermented material, with a VSS concentration of 34,500 mg/l, was fed continuously into the wet oxidation pressure vessel. The rate of addition was based on attaining a theoretical 2 hour retention time for liquid within the reactor. Oxygen concentration within the reactor was under automatic control, starting with a 20 bar overpressure of oxygen (BOC NZ Ltd—zero grade). Total pressure in the wet oxidation pressure vessel was maintained at 45 bar throughout. Temperature was controlled at 220° C. throughout.

### Analysis

The water quality parameters, total suspended solids (TSS), volatile suspended solids (VSS), dissolved organic carbon (DOC), total chemical oxygen demand (COD), soluble chemical oxygen demand (SCOD) and particulate chemical oxygen demand (PCOD) were measured following standard analytical procedures (APHA, 1998).

The volatile fatty acids (VFA) were determined by a method involving pH correction with formic acid, followed by capillary gas chromatography with flame ionisation detection (GC-FID). The column used was a 30 m×0.53 μm ID Nukol™ ramped from 40 to 150 W C. Butan-1-ol solution was used as the internal standard. The total residual organic carbon concentration (TOC) in filtered samples was also measured with a TOC analyser (Elementar High TOC II).

Nitrite (NO 2-N), nitrate (NO 3-N), total Kjeldahl nitrogen (TKN) and dissolved reactive phosphorus (DRP; as PO 4-P) species were determined according to standard methods (APHA, 1998).

### Results

TSS destruction was 15% after fermentation and 78% after wet oxidation. VSS destruction was 19% destruction after fermentation and 89% after wet oxidation.

No significant total COD reduction was observed after fermentation. Approximately 50% reduction of total COD was observed after wet oxidation. Soluble COD (sol COD) increased across each process stage.

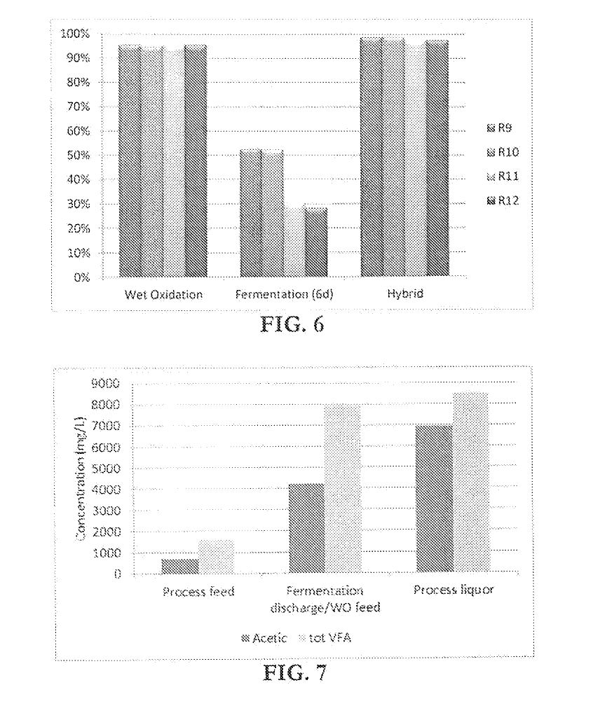

Soluble organics, as measured by acetic acid, total VFA, soluble COD and dissolved organic carbon (DOC) increased across each process stage, as shown in FIGS. 7 and 8.

Soluble nitrogen as ammoniacal N (NH4-N) and dissolved kjeldahl N (DKN) increased across each process stage. Soluble phosphorus (sol P) increased across fermentation but decreased across the whole process due to action within wet oxidation stage.

The 4-day retention time and the anaerobic conditions used during fermentation resulted in suppression of methane production. For the fermentation conditions described above, an average biogas yield of 0.027 m³/kgVS added was observed, of which less than ⅕th was methane, and mean total COD in the fermentation feed was measured to be 67,425 mg/l, whilst mean fermentation discharge total COD was 68,983 mg/l. This result indicated minimal organic carbon loss as gaseous CO₂ or CH, during fermentation.

### Example 5

This example demonstrates the operation of the method described herein and describes the impact of fermentation VFA formation within a batch wet oxidation, at pilot plant scale and compares with VFA formation within a batch wet oxidation using non-fermented feedstock.

### Method

### Pilot Plant Fermentation

A 2000 L total volume (1000 L working volume) pilot plant fermentation reactor was used. This was continuously stirred, mechanically, and the temperature maintained at 35° C. via a water jacket. Anaerobic conditions were maintained throughout and nitrogen was added to the headspace as required during sludge discharge to maintain a positive pressure. The pH was recorded and automatically controlled via acid (H₂SO₄) or alkali (NaOH) addition.

Biosolids from a municipal biological nutrient removal wastewater treatment plant were automatically fed, three times daily, into the fermentation reactor and fermented material was discharged. Across the 6 month course of fermenter operation described for these experiments, a number of fermentation parameters were adjusted, including feed solids concentration (4-6% by weight), solids retention time (3.5-7 d) and pH control (5.5-6.2).

### Biosolids Samples for Wet Oxidation

Samples were taken directly from the beltpress of the municipal wastewater treatment plant or from the pilot plant fermentation of the same solids. These were diluted with water to provide a starting concentration in the wet oxidation reactor of 1-3% (by weight).

### Pilot Plant Wet Oxidation

A 200 L total reactor volume (80 L working volume) pilot plant wet oxidation pressure vessel was used. This was mixed via liquid recirculation and gas recirculation pumps. Biosolids material, was added in batch form into the wet oxidation pressure vessel, to starting concentrations of between 10-25 g/l. The pressure vessel was raised to a working temperature of 220° C. via external heat exchange.

Oxygen concentration within the reactor was under semi-automatic control, starting with a 20 bar overpressure of oxygen (BOC NZ Ltd—zero grade). Depending on the experiment, total pressure in the wet oxidation pressure vessel was maintained at 40-50 bar throughout the experimental period.

The batch experiments were conducted over a period of 5 hours, with sampling from the reaction vessel for the water quality parameters, total suspended solids (TSS), volatile suspended solids (VSS), dissolved organic carbon (DOC), total chemical oxygen demand (COD), soluble chemical oxygen demand (SCOD) and particulate chemical oxygen demand (PCOD), following standard analytical procedures (APHA, 1998). The volatile fatty acids (VFA) were determined by a method involving pH correction with formic acid, followed by capillary gas chromatography with flame ionisation detection (GC-FID). The column used was a 30 m×0.53 μm ID Nukol™ ramped from 40 to 150 W C. Butan-1-ol solution was used as the internal standard. The total residual organic carbon concentration (TOC) in filtered samples was also measured with a TOC analyser (Elementar High TOC II).

The results from two separate batch wet oxidations on unfermented biosolids are described and are referred to below as “unfermented”.

The results from three separate batch wet oxidations on fermented biosolids are described and are referred to below as “fermented”.

### Results and Discussion

The individual wet oxidation runs for each sample type (fermented or unfermented) have been combined in the analysis.

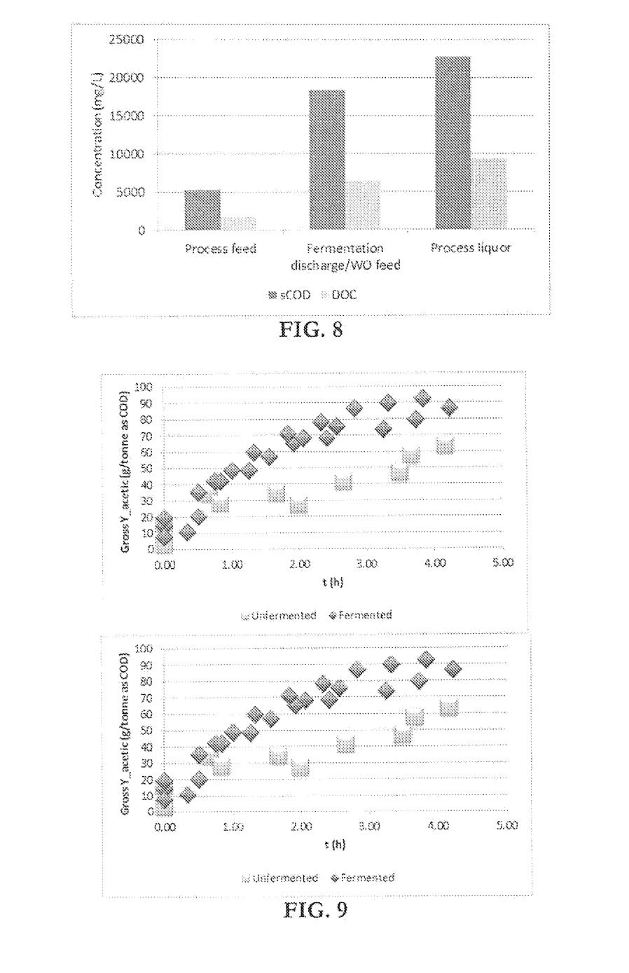

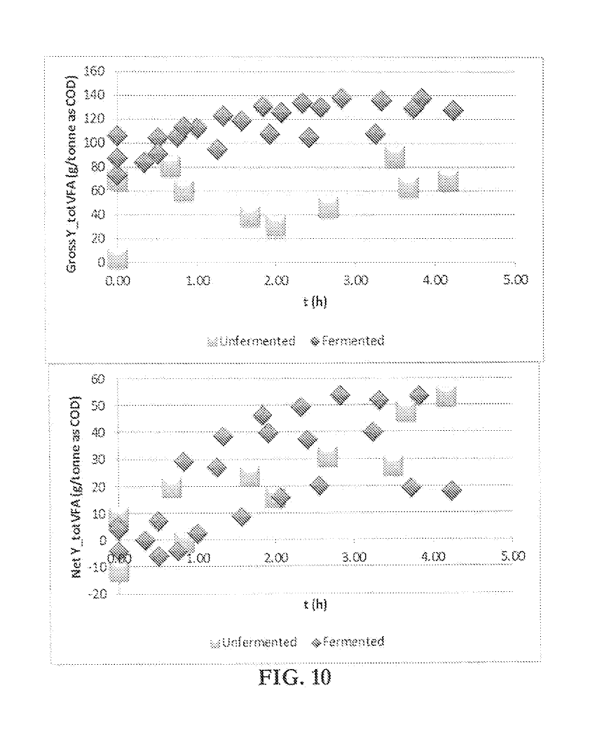

FIGS. 9 and 10 provide an analysis of the acetic acid and total VFA (sum of COD equivalents of acetic, propionic, n-butyric, iso-butyric, pentanoic, hexanoic acids, ethanol and methanol) yield for the two sample types. Results are presented as yields, on a COD equivalent basis. The time is set for t=0 being the sample time at which the wet oxidation reaction reached the range 160-190° C., a range in which significant COD conversions were observed to begin.

Yield Calculations were as Follows.

$$Gross yield = \frac{{[ analyte ]}_{time = t}}{{[ C O D ]}_{t = 0}}$$

The gross yield calculation includes any impact of the fermentation stage on yields.

$$Net yield = \frac{{[ analyte ]}_{t} - {[ analyte ]}_{t = 0}}{{[ C O D ]}_{t = 0}}$$

The net yield calculation describes yield within wet oxidation only.

Referring to FIG. 9, acetic acid yields demonstrate a clear yield benefit of the fermented biosolids, for both gross (two stage system) and net (wet oxidation stage only) yields.

Referring to FIG. 10, these yields sum the overall impact of VFA degradation within wet oxidation, for all but the acetic acid molecule. Gross VFA yields are higher for the fermented sample, reflecting the impact of fermentation of solids to VFA in the fermentation stage. Net yields across the wet oxidation stage alone were variable for the fermented sample type. Investigation of the individual wet oxidation trials indicated that one of the samples used within the experiment had significantly higher propionic acid present prior to wet oxidation, a result of its production in the fermentation stage. This propionic was subject to degradation within the wet oxidation stage, lowering the observed net yield for this run relative to the other trials. This data from this sample is visible as the lower band of fermented sample data points.

Accounting for the high propionic sample described above, the net yields through wet oxidation were not greatly different between fermented and unfermented sample types.

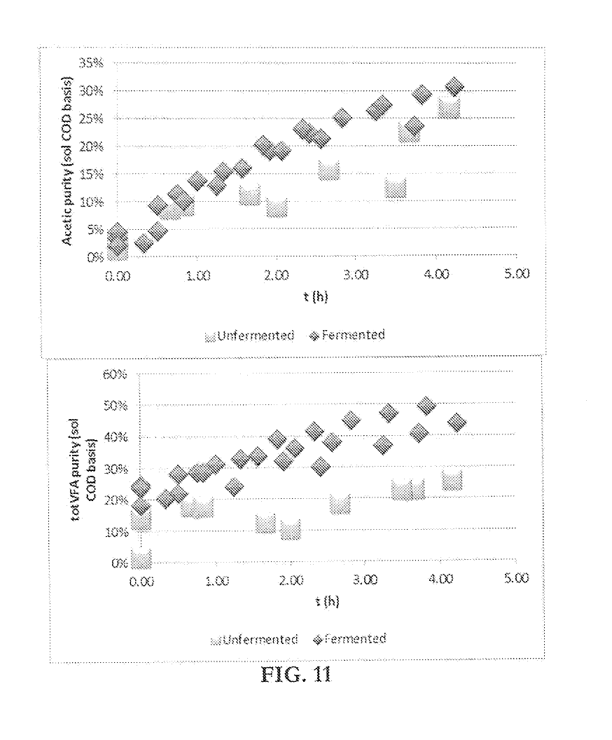

FIG. 11 presents the purity of acetic acid and total VFA across the course of the wet oxidation, as a fraction of the soluble COD present within the sample. The low purity for both samples at t=0 reflects the significant amount of solids solubilisation which occurred on transitioning from ambient to wet oxidation start temperature (defined here as T=160-185° C.). For acetic acid, the purity of the fermentation sample type was higher than that of the unfermented samples, up to t=4 hrs, where the differences diminish. This differs to the total VFA purity, where fermented solids produced increasingly higher purity across the batch experimental time.

### INDUSTRIAL APPLICATION

The processes of the invention have application in the treatment of waste biomass, such as municipal and industrial waste biomass.

Those persons skilled in the art will understand that the above description is provided by way of illustration only and that the invention is not limited thereto.

### REFERENCES

APHA, 1998. Standard methods for the examination of water and wastewater. American Public Health Association, USA.
Appels, L., Baeyens, J., Degreve, J., Dewil, R., 2008. Principles and potential of the anaerobic digestion of waste-activated sludge. Progress in Energy and Combustion Science, 34, 755-781.
Bhargava, S. K., Tardio, J., Prasad, J., ger, K., Akolekar, D. B., Grocott, S. C., 2006. Wet oxidation and catalytic wet oxidation. Industrial and Engineering Chemistry Research, 45, 1221-1258.
Kraemer, J. T., Bagley, D. M., 2007. Improving the yield from fermentative hydrogen production. Biotechnology Letters, 29, 685-695.
Mishra, V. S., Mahajani, V. V., Joshi, J. B., 1995. Wet air oxidation. Industrial and Engineering Chemistry Research, 34, 2-48.
Wang, J., Wan, W., 2009. Factors influencing fermentative hydrogen production: A review. International Journal of Hydrogen Energy, 34, 799-811.

What we claim is:

1. A process for the treatment of biomass comprising subjecting biomass to microbial digestion, preferably anaerobic microbial digestion to produce volatile fatty acids and/or solvents followed by wet oxidation to reduce biosolid volume while retaining or increasing the concentration of the volatile fatty acids and/or solvents.

2. A process for the treatment of biomass comprising
(1) subjecting biomass to microbial digestion, preferably anaerobic microbial digestion under conditions so as to convert at least a portion of the organic biomass to volatile fatty acids and/or solvents while leaving at least some of the organic biomass in the form of biosolids or unconverted organic material to create a mixture of biosolids, unconverted organic biomass and volatile fatty acids and/or solvents, and
(2) subjecting the mixture to wet oxidation thereby reducing biosolid volume and producing a resulting mixture under conditions that do not result in the mass destruction of the volatile fatty acids and/or solvents.

3. A process for the treatment of biomass comprising
(1) subjecting biomass to microbial digestion, preferably anaerobic microbial digestion by contacting biomass with one or more microorganisms under conditions that promote acidogenesis while retarding methanogenesis to produce a mixture comprising
(a) volatile fatty acids and/or solvents such as short chain (C1 to C7) fatty acids, short chain (C1 to C7) alcohols, short chain (C1 to C7) ketones or any mixture of any two or more thereof, and
(b) undigested biomass, and
(2) subjecting at least a portion of the mixture to wet oxidation under conditions to reduce the volume of the undigested biomass while maintaining or increasing the concentration of the volatile fatty acids and/or solvents that are present, the wet oxidation conditions optionally comprising in one embodiment a residence time of less than about 120 minutes.

4. The process of any one of claims 1 to 3 wherein the biomass comprises a hydrocarbon source.

5. The process of any one of claims 1 to 3 wherein the biomass comprises a hydrocarbon source selected from the group comprising biological material, organic matter, plant matter, animal matter, waste material, organic waste material, plant waste material, animal waste material, dairy processing wastewater, abattoir wastewater, abattoir waste material, food processing wastewater, food processing waste material, wood pulp, lignocellulose pulp, pulp processing wastewater, pulp processing waste material, paper processing wastewater, paper processing waste material, municipal waste material, municipal wastewater, solids from municipal wastewater, lignocellulosic biomass, wastewater from lignocellulosic biomass processing, biosolid waste material from lignocellulosic biomass processing, or any combination of any two or more thereof.

6. The process of any one of claims 1 to 5 wherein the solids content of the biomass is at least about 0.5 to about 70% by weight.

7. The process of any one of claims 1 to 6 wherein the biomass comprises one or more microorganisms.

8. The process of any one of claims 1 to 6 wherein the biomass is substantially free of microorganisms.

9. The process of any one of claims 1 to 8 wherein the process comprises applying conditions such that the microbial digestion of the organic biomass generates volatile fatty acids and/or solvents but minimises methanogenesis or other further digestion of the volatile fatty acids and/or solvents.

10. The process of any one of claims 1 to 9 wherein the microbial digestion conditions comprise a temperature of up to about 1 to about 50° C.

11. The process of any one of claims 1 to 10 wherein the microbial digestion conditions comprise a pH of about 4 to about 6.4 or a pH of about 7.3 to about 10.

12. The process of any one of claims 1 to 11 wherein the microbial digestion conditions comprise a volatile suspended solids content of about 0.5 to about 10 g/L.

13. The process of any one of claims 1 to 12 wherein the microbial digestion conditions comprise a digestion time of up to about 0.5 to about 20 days.

14. The process of any one of claims 1 to 13 wherein the microbial digestion is continued until the concentration of volatile fatty acids and/or solvents in the digestion medium reaches a maximum.

15. The process of any one of claims 1 to 13 wherein the microbial digestion is continued until the concentration of volatile fatty acids and/or solvents is at least about 100 to about 250 mg/gVSS.

16. The process of any one of the preceding claims wherein the process provides a gross yield of acetic acid that is at least about 10% to about 100% or more greater than with wet oxidation of unfermented biosolids.

17. The process of any one of the preceding claims wherein the process provides a gross yield of acetic acid over the first 1, 2, 3, 4, or 5 hours of oxidation that is at least about 10% to about 100% or more greater than with wet oxidation of unfermented biosolids.

18. The process of any one of the preceding claims wherein the process provides a gross yield of volatile fatty acids that is at least about 10% to about 85% or more greater than with wet oxidation of unfermented biosolids.

19. The process of any one of the preceding claims wherein the process provides a gross yield of volatile fatty acids over the first 1, 2, 3, 4, or 5 hours of oxidation that is at least about 10% to about 85% or more greater than with wet oxidation of unfermented biosolids.

20. The process of any one of the preceding claims wherein the process provides acetic acid purity, or volatile fatty acid purity, or both that is at least about 10, 20 or 30% greater than with wet oxidation of unfermented by solids.

21. The process of any one of the preceding claims wherein the process provides an increase in the rate of production of acetic acid or of total volatile fatty acids (VFA), or both that is at least 10%, 20%, 30%, 40%, 50% faster that the rate of production of acetic acid or total volatile fatty acids, or both from unfermented solids under similar wet oxidation conditions.

22. The process of any one of the preceding claims wherein the process provides an increase in the rate of production of acetic acid or of total volatile fatty acids, or both over the first 1, 2, 3, 4, or 5 hours of wet oxidation that is at least 10%, 20%, 30%, 40% or 50% or more faster than the rate of production of acetic acid or total volatile fatty acids, or both from unfermented solids under similar wet oxidation conditions.

23. A process of any one of claims 1 to 22 wherein the microbial digestion conditions or the one or more microorganisms comprises one or more mixed cultures or one or more monocultures of bacteria or algae or a combination thereof.

24. A process of any one of claims 1 to 23 wherein the microbial digestion conditions or the one or more microorganisms comprises one or more acidogenic microorganisms selected from Acetobacterium, Aeromonas, Clostridia, Klebsiella, Moorella and Ruminococcus, and any combination of any two or more thereof.

25. A process of any one of claims 1 to 24 wherein the microbial digestion conditions or the one or more microorganisms comprises one or more acidogenic microorganisms selected from Acetobacterium spp., Aeromonas spp., Clostridia spp., Klebsiella spp., Moorella spp. and Ruminococcus spp., Acetobacterium woodii, Clostridium thermoaceticum, Clostridium thermolacticum, Clostridium ljungdahlii, Clostridium acetobutylicum, Clostridium formicaceticum, Clostridium glycolicum, Moorella thermoautotrophica, and Ruminococcus productus, and any combination of any two or more thereof.

26. A process of any one of claims 1 to 25 wherein the microbial digestion conditions or the one or more microorganisms comprises one or more acetogenic microorganisms.

27. A process of any one of claims 1 to 26 wherein the microbial digestion conditions or the one or more microorganisms comprises one or more microorganisms selected from Acetobacterium, Clostridium, Moorella and Ruminococcus, and any combination of any two or more thereof.

28. A process of any one of claims 1 to 27 wherein the microbial digestion conditions or the one or more microorganisms comprises one or more microorganisms selected from Acetobacterium woodii, Clostridium thermoaceticum, Clostridium thermolacticum, Clostridium ljungdahlii, Clostridium acetobutylicum, Clostridium formicaceticum, Clostridium glycolicum, Moorella thermoacetica, Moorella thermoautotrophica and Ruminococcus productus, and any combination of any two or more thereof.

29. A process of any one of claims 1 to 28 wherein the microbial digestion conditions or the one or more microorganisms comprises one or more microorganisms selected from one or more acidogenic or acetogenic algae

30. A process of any one of claims 1 to 25 wherein the microbial digestion conditions are substantially free of hydrogen gas.

31. A process of any one of claims 1 to 26 wherein the solids content of the mixture resulting from microbial digestion is, or is diluted or dewatered to, about 0.5 to about 10% by weight.

32. A process of any one of claims 1 to 26 wherein the wet oxidation conditions comprise a temperature of up to the critical point of water, about 100 to about 374° C.

33. A process of any one of claims 1 to 27 wherein the wet oxidation conditions comprise an oxidant.

34. A process of claim 28 wherein the oxidant concentration is at least about 0.5. 0.75, 1, 1.5 or 2 times the stoichiometric amount required for complete oxidation of the organic material in the mixture entering the wet oxidation stage.

35. A process of any one of claims 1 to 29 wherein the wet oxidation conditions comprise a residence time of about 5 to about 180 minutes.

36. A process of any one of claims 1 to 30 wherein the wet oxidation conditions reduce the volume of biosolids by at least about 60 to 99%.

37. A process of any one of claims 1 to 32 wherein an amount of liquid from wet oxidation is added to an amount of the mixture before the mixture is subjected to wet oxidation.

38. A process of any one of claims 1 to 30 wherein the process further comprises separating at least one of the volatile fatty acids or solvents from the mixture following wet oxidation.

39. A process of any one of claims 1 to 31 wherein the process further comprises separating ammonium from the mixture following wet oxidation.

40. A process of any one of claims 1 to 32 wherein the process further comprises separating a precipitated phosphorus-containing compound from the mixture following wet oxidation.

41. A process of producing a fuel or fuel precursor, the process comprising processing at least one of the separated volatile fatty acids or solvents produced by a method of any one of claims 1 to 33 into a fuel or fuel precursor.

42. A process of claim 34 wherein the fuel or fuel precursor comprises alcohol.
